(19) United States
(12) Patent Application Publication
(10) Pub. No.: US 2022/0146524 A1
(43) Pub. Date: May 12, 2022

(54) BIOLOGICAL INFORMATION MEASUREMENT DEVICE AND BIOLOGICAL INFORMATION MEASUREMENT METHOD

(71) Applicant: PHC Holdings Corporation, Tokyo (JP)

(72) Inventors: Noriyoshi TERASHIMA, Ehime (JP); Masataka NADAOKA, Ehime (JP)

(21) Appl. No.: 17/440,316

(22) PCT Filed: Mar. 16, 2020
(86) PCT No.: PCT/JP2020/011458

§ 371 Date: Sep. 17, 2021

(30) Foreign Application Priority Data
Apr. 5, 2019 (JP) 2019-072615

(51) Int. Cl. G01N 33/66; G16H 10/40; G16H 10/60

(52) U.S. Cl. CPC G01N 33/66; G16H 10/60; G16H 10/40

(57) ABSTRACT

A biological information measurement device comprises a sensor insertion port, a measurement unit, a communication unit, and a control unit. In the sensor insertion port is mounted a sensor for measuring biological information about a patient. The measurement unit is connected to the sensor mounted in sensor insertion port and measures the blood glucose level. The communication unit acquires information about the patient whose biological information is being measured. The control unit determines the risk status of the patient on the basis of the information about the patient acquired by the communication unit, and issues a warning about the measurement of the biological information of the patient with the measurement unit on the basis of the determination result.

### TECHNICAL FIELD

The present invention relates, for example, to a biological information measurement device and a biological information measurement method for measuring biological information such as blood glucose level.

### BACKGROUND ART

In a conventional biological information measurement device, a biosensor for measuring blood glucose level or other such biological information is mounted on a sensor mounting portion, voltage is supplied from a measurement voltage supply means between first and second connectors, and the current flowing between the first and second connectors is measured by a current measurement means, thereby measuring the blood glucose level or other such biological information, or an optical principle is utilized to detect the reaction of a different biosensor and measure CRP (C-reactive protein) or the like. There are also devices that examine the exogenous coagulation activation mechanism by measuring the prothrombin time, in which the change in blood viscosity is physically measured.

For example, Patent Literature 1 discloses a system for checking the correlation between a disease name and a medical procedure in an online environment for confirming the validity of a medical procedure order, on the basis of the patient's inputted complaint and symptoms.

### CITATION LIST

### Patent Literature

Patent Literature 1: JP H8-101871 A

### SUMMARY

### Technical Problem

However, the following problem is encountered with the above-mentioned conventional system.

With the system disclosed in the above publication, it is possible to confirm the validity of a subsequent medical procedure on the basis of the inputted complaint and symptoms of the patient, but no consideration whatsoever is given to dealing with the measurement procedure when measuring biological information.

It is an object of the present invention to provide a biological information measurement device and a biological information measurement method with which a warning about the measurement of biological information can be issued on the basis of a patient's medical history and symptoms, medications being taken, and other such information.

### Solution to Problem

The biological information measurement device according to the first invention comprises a sensor mounting unit, a measurement unit, an information acquisition unit, and a control unit. A sensor for measuring biological information about the patient is mounted on the sensor mounting unit. The measurement unit is connected to the sensor mounted to the sensor mounting unit, and measures the biological information. The information acquisition unit acquires information about the patient for whom the biological information is measured. The control unit determines the risk status of the patient on the basis of the information about the patient acquired by the information acquisition unit, and issues a warning about the measurement of biological information about the patient with the measurement unit on the basis of the determination result.

In a biological information measurement device that measures biological information such as a blood glucose level, for example, information is acquired about the patient for whom biological information is to be measured, and a warning about the measurement of the patient's biological information is issued on the basis of the risk status of the patient determined according to the information about the patient.

Here, information about the patient includes, for example, one or more of the following: the medical history of the patient, medications being taken, current symptoms, risk level, age, diet, and supplements. Also, the information acquisition unit that acquires information about the patient includes an input unit for directly inputting information about the patient, a communication unit for acquiring information about the patient from an external server device or the like, and so forth. Furthermore, the information about the patient may be stored in a memory unit within the biological information measurement device, or may be stored in an external device such as a server device.

Also, the determination of the risk status of the patient includes, for example, determination of the extent of dehydration, which may affect the biological information measurement result, on the basis of the information about the patient. That is, if the patient is dehydrated, there may be variance in the blood glucose level or other such biological information measurement result, and the measurement accuracy may decrease.

Also, the warning about measuring biological information includes, for example, control causing the display unit to display a message confirming whether or not to measure biological information, a message indicating that the biological information measurement result is a reference value, a message recommending multiple measurements, or a message recommending consultation with the doctor in charge. These warnings may be issued by voice output or optical output, and are not limited to a text display on the display unit.

Consequently, if the risk status of the patient is determined on the basis of the patient's medical history, medications being taken, and other such information about the patient, in the event that there is a risk that the error in the measured value will increase, depending on the risk status of the patient, for example, a warning that takes into account the risk of error can be issued.

As a result, a warning about the measurement of biological information can be issued on the basis of information such as the patient's medical history and symptoms, and the medications being taken.

The biological information measurement device according to the second invention is the biological information measurement device according to the first invention, wherein the information about the patient includes one or more of the following: the medical history of the patient, medications being taken, current symptoms, risk level, age, diet, and supplements.

One or more of the following are acquired as information about the patient: the patient's medical history, medications being taken, current symptoms, risk level, age, diet, and supplements.

Here, the patient risk level is a level indicating the severity of illness of the patient, as scored by the ICU scoring system or the like, and includes, for example, the APACHE (Acute Physiology And Chronic Health Evaluation) II score, the MODS (Multiple Organ Dysfunction) score, and so forth.

Consequently, by determining the risk status of the patient on the basis of the information about the patient, it is possible to issue a warning about the measurement of biological information on the basis of the information such as the patient's medical history and symptoms, medications being taken, and so forth.

The biological information measurement device according to the third invention is the biological information measurement device according to the first or second invention, further comprising a memory unit that stores information about the patient.

Here, information about the patient is stored in a memory unit provided within the device.

This allows information about a patient to be taken out of the memory unit and easily used for determining the risk status of the patient.

The biological information measurement device according to the fourth invention is a biological information measurement device according to any of the first to third inventions, further comprising a communication unit that transmits and receives information about the patient to and from an external device.

Here, a communication unit is provided that transmits and receives necessary information to and from an external device in which information about the patient and the like are stored.

Consequently, necessary information such as information about the patient is acquired from an external device such as a server device, and is transmitted to the control unit, and this allows a warning to be issued about the measurement of biological information on the basis of information such as the patient's medical history and symptoms, medications being taken, and so forth.

The biological information measurement device according to the fifth invention is the biological information measurement device according to the fourth invention, wherein the communication unit transmits and receives information about the patient to and from a server device provided externally.

Here, a server device is used as the external device.

Consequently, necessary information such as information about the patient stored in the server device is acquired and transmitted to the control unit, which allows a warning to be issued about the measurement of biological information on the basis of information such as the patient's medical history and symptoms, medications being taken, and so forth, without having to provide a large-capacity memory means in the device.

The biological information measurement device according to the sixth invention is the biological information measurement device according to any of the first to fifth inventions, further comprising a display unit that displays the measurement result from the measurement unit.

Here, the display unit displays biological information measurement results and the like.

This allows the user to easily recognize the biological information measurement results.

The biological information measurement device according to the seventh invention is the biological information measurement device according to the sixth invention, wherein the control unit controls the display unit so as to display to a measurer a message asking whether or not to measure the biological information of the patient, on the basis of the determination result.

Here, the display unit displays a message asking whether or not to measure the biological information, depending on the risk status of the patient determined on the basis of information about the patient.

Consequently, the measurer can decide whether or not to measure the biological information after first recognizing the risk entailed by the measurement of biological information about the patient that is about to be performed, that this measurement may have an influence such as increasing the measurement error, for example.

The biological information measurement device according to the eighth invention is the biological information measurement device according to the sixth invention, wherein the control unit controls the display unit so as to display to a measurer a message telling to measure the biological information of the patient as a reference value, on the basis of the determination result.

Here, the display unit displays a message indicating that the biological information measurement result is measured as a reference value, depending on the risk status of the patient determined on the basis of information about the patient.

Consequently, the measurer can recognize that a reference value is being displayed after first recognizing the risk entailed by the measurement of biological information about the patient that is about to be performed, that this measurement may have an influence such as increasing the measurement error, for example.

The biological information measurement device according to the ninth invention is the biological information measurement device according to the sixth invention, wherein the control unit controls the display unit so as to display a measurer the number of measurements when measuring the biological information about the patient, on the basis of the determination result.

Here, the display unit displays a message that presents the number of measurements of the biological information, depending on the risk status of the patient determined on the basis of information about the patient.

This allows the measurer increase the number of times the biological information is measured, and refer to the average value thereof, after first recognizing the risk entailed by the measurement of biological information about the patient that is about to be performed, that this measurement may have an influence such as increasing the measurement error, for example.

The biological information measurement device according to the tenth invention is the biological information measurement device according to any of the first to ninth inventions, further comprising a reading unit that reads information about the patient, the measurer, and the sensor.

Here, for example, a reading unit is provided for reading barcodes attached to the wrist of the patient, the name tag of the measurer, the outer peripheral surface of the sensor bottle containing the sensor, and the like.

Consequently, when measuring biological information, information about the patient can be easily acquired by reading the barcode wrapped around the patient's wrist, for example.

The biological information measurement method according to the eleventh invention comprises an information acquisition step, a determination step, and a warning step. The information acquisition step involves acquiring information about a patient whose biological information is to be measured. The determination step involves determining the risk status of the patient on the basis of the information about the patient acquired in the information acquisition step. The warning step involves issuing a warning about the measurement of biological information about the patient, on the basis of the determination result in the determination step.

In a biological information measurement device that measures biological information such as blood glucose level, for example, information is acquired about the patient for whom biological information is to be measured, and a warning about measurement of the patient's biological information is issued on the basis of the risk status of the patient determined according to the information about the patient.

Here, the information about the patient includes, for example, one or more of the following: the patient's medical history, medications being taken, current symptoms, risk level, age, diet, and supplements. Also, the information acquisition unit for acquiring information about the patient includes an input unit for directly inputting information about the patient, a communication unit for acquiring information about the patient from an external server device or the like, and so forth.

Also, the determination of the risk status of the patient includes, for example, determination of the extent of dehydration, which may affect the biological information measurement result, on the basis of the information about the patient. That is, if the patient is dehydrated, there may be variance in the blood glucose level or other such biological information measurement result, and the measurement accuracy may decrease.

Also, the warning about measuring biological information includes, for example, displaying a message confirming whether or not to measure biological information, displaying a message indicating that the biological information measurement result is a reference value, displaying a message recommending multiple measurements, displaying a message recommending consultation with the doctor in charge, and so forth. These warnings may be issued by voice output or optical output, and are not limited to a text display on the display unit.

Consequently, if the risk status of the patient is determined on the basis of the patient's medical history, medications being taken, and other such information about the patient, in the event that there is a risk that the error in the measured value will increase, depending on the risk status of the patient, for example, a warning that takes into account the risk of error can be issued.

As a result, a warning about the measurement of biological information can be issued on the basis of information such as the patient's medical history and symptoms, and the medications being taken.

The biological information measurement method according to the twelfth invention is the biological information measurement method according to the eleventh invention, wherein, in the warning step, a message asking whether or not to measure biological information about the patient is displayed on the display unit to a measurer on the basis of the determination result in the determination step.

Here, the display unit displays a message asking whether or not to measure the biological information depending on the risk status of the patient determined on the basis of information about the patient.

Consequently, the measurer can decide whether or not to measure the biological information after first recognizing the risk entailed by the measurement of biological information about the patient that is about to be performed, that this measurement may have an influence such as increasing the measurement error, for example.

The biological information measurement method according to the thirteenth invention is the biological information measurement method according to the eleventh invention, wherein, in the warning step, a message telling to measure the biological information about the patient as a reference value is displayed on the display unit to a measurer on the basis of the determination result in the determination step.

Here, the display unit displays a message indicating that the measurement result of the biological information is measured as a reference value, depending on the risk status of the patient determined on the basis of information about the patient.

Consequently, the measurer can recognize that a reference value is being displayed after first recognizing the risk entailed by the measurement of biological information about the patient that is about to be performed, that this measurement may have an influence such as increasing the measurement error, for example.

The biological information measurement method according to the fourteenth invention is the biological information measurement method according to the eleventh invention, wherein, in the warning step, the display unit displays the number of measurements when measuring the biological information about the patient to a measurer, on the basis of the determination result in the determination step.

Here, a message presenting the number of measurements of biological information is displayed on the display unit, depending on the risk status of the patient determined on the basis of information about the patient.

This allows the measurer increase the number of times the biological information is measured, and refer to the average value thereof, after first recognizing the risk entailed by the measurement of biological information about the patient that is about to be performed, that this measurement may have an influence such as increasing the measurement error, for example.

### Advantageous Effects

With the biological information measurement device according to the present invention, a warning about the measurement of biological information can be issued on the basis of information such as a patient's medical history and symptoms, medications being taken, and so forth.

### BRIEF DESCRIPTION OF DRAWINGS

FIG. 1 is a diagram showing a state in which a blood glucose level is measured using the biological information measurement device according to an embodiment of the present invention;

FIG. 2 is a control block diagram of the biological information measurement device in FIG. 1;

FIG. 3 is a flowchart showing the flow of processing to warn about measuring the blood glucose level with the biological information measurement device in FIG. 2;

FIGS. 4A and 4B are diagrams showing a display example displayed on the display unit when the measurer ID is read by a barcode reader during the processing in FIG. 3;

FIGS. 5A and 5B are diagrams showing a display example displayed on the display unit when the patient ID is read by a barcode reader during the processing in FIG. 3;

FIGS. 6A and 6B are diagrams showing a display example displayed on the display unit when the sensor ID is read by a barcode reader during the processing in FIG. 3;

FIGS. 7A to 7D are diagrams showing a display example displayed on the display unit during the processing in FIG. 3;

FIGS. 8A to 8C are diagrams showing a display example displayed on the display unit during the processing in FIG. 3;

FIGS. 9A to 9C are diagrams showing a display example displayed on the display unit as a result of the processing in FIG. 3;

FIG. 10 is a flowchart showing the flow of processing to warn about measurement of blood glucose level with the biological information measurement device according to another embodiment of the present invention;

FIG. 11 is a flowchart showing a flow of processing in the latter part of the flowchart of FIG. 10:

FIGS. 12A and 12B are diagrams showing a display example displayed on the display unit during the processing in FIGS. 10 and 11; and

FIGS. 13A to 13D are diagrams showing a display screen on which information about a patient is directly inputted in the biological information measurement device according to yet another embodiment of the present invention.

### DESCRIPTION OF EMBODIMENTS

### Embodiment 1

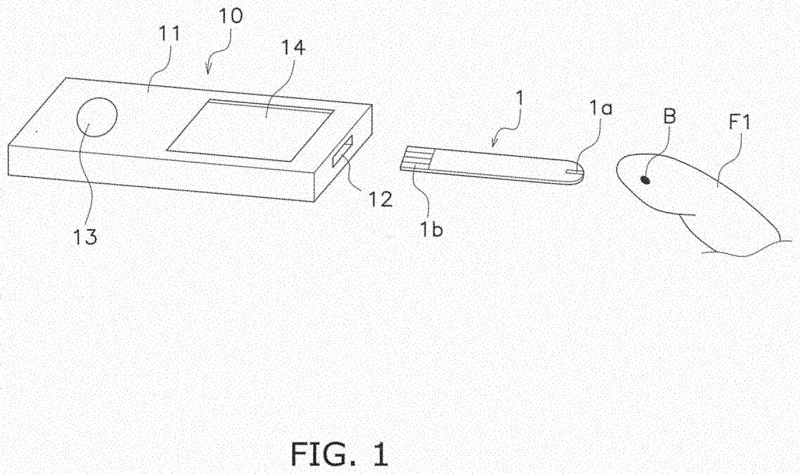

The biological information measurement device according to an embodiment of the present invention will now be described with reference to FIGS. 1 to 9C.

The biological information measurement device 10 in this embodiment is, for example, a blood glucose sensor that measures the blood glucose level (biological information) in a state in which a sensor 1, on which blood B from a puncture made in a finger F1 of a patient has been deposited, is inserted. The biological information measurement device 10 is mainly used in hospitals, nursing care facilities, and so forth in which the blood glucose levels of a plurality of patients are measured using a single biological information measurement device 10.

Here, as shown in FIG. 1, the sensor 1 mounted in the sensor insertion port 12 of the biological information measurement device 10 has a deposition port 1a and an electrode portion 1b.

The deposition port 1a is provided at a first end of the sensor 1. When the blood B that has come out of the patient's finger F1 is deposited in the deposition port 1a, the blood B moves by capillary action in a specific direction along the supply path within the sensor 1, and reacts with a reagent layer (not shown) provided inside the sensor 1.

The electrode portion 1b is provided at a second end on the side of the biological information measurement device 10 that is inserted into the sensor insertion port 12. The above-mentioned reagent layer contains an oxidoreductase and an electron acceptor, and is disposed in the sensor 1 so as to cover part of the electrode portion 1b. When the electrode portion 1b is inserted into the sensor insertion port 12, the sensor 1 and the biological information measurement device 10 are electrically connected, and the current produced by the electrochemical oxidation of the electron acceptor contained in the reagent layer as a result of the reaction of between the blood B and the reagent layer is measured.

This makes it possible to measure the glucose concentration in the blood (blood glucose level) using a biological information measurement device.

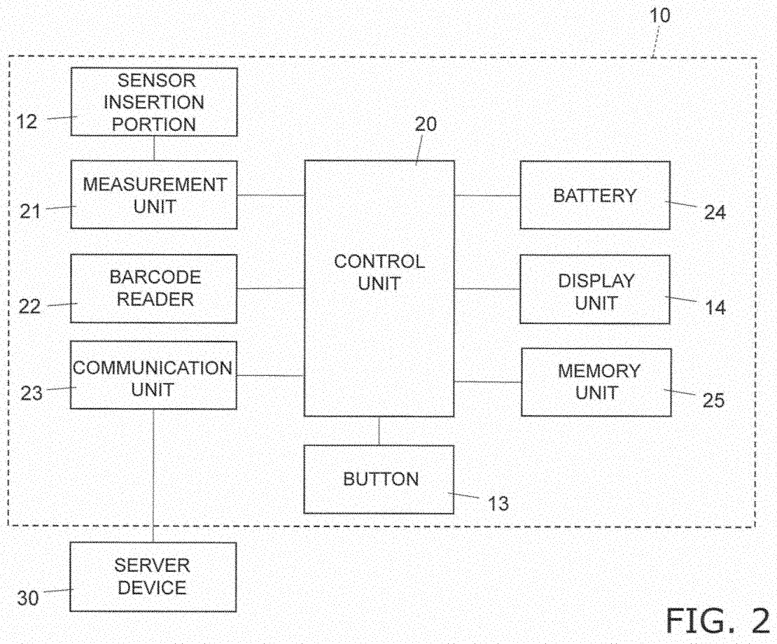

As shown in FIG. 1, the biological information measurement device 10 comprises a main body 11, a sensor insertion port (sensor mounting portion) 12, a button 13, a display unit 14, a control unit 20 (see FIG. 2), a measurement unit 21, a barcode reader (reading unit) 22, a communication unit 23, a battery 24, and a memory unit 25 (see FIG. 2).

The main body 11 is the housing portion of the biological information measurement device 10, and internally holds the control unit 20, the measurement unit 21, the barcode reader 22, the communication unit 23, the battery 24, and the memory unit 25 shown in FIG. 2.

The sensor insertion port 12 is an opening formed in the end surface of the main body 11, and the above-mentioned sensor 1 is mounted therein.

The button 13 is an operation unit that is operated upon starting measurement of the blood glucose level, etc., and is provided on the surface of the main body 11 where the display unit 14 is provided.

The display unit 14 is a liquid crystal display panel provided on the surface of the main body 11 on the sensor insertion port 12 side, and displays, for example, a blood glucose measurement result, a warning message about the measurement of blood glucose level (discussed below), and the like.

As shown in FIG. 2, the control unit 20 is connected to the measurement unit 21, the barcode reader 22, the communication unit 23, the button 13, the battery 24, the display unit 14, and the memory unit 25.

As shown in FIG. 2, the measurement unit 21 is connected to a connector (not shown) of the sensor insertion port 12. The measurement unit 21 measures the current obtained by the electrochemical oxidation of the electron acceptor contained in the reagent layer as a result of the reaction between the blood B and the reagent layer in the electrode portion 1b of the sensor 1 connected to the sensor insertion port 12.

The barcode reader 22 is provided, for example, on the back surface side of the main body 11, reads the patient ID, user ID, sensor bottle ID, etc., during measurement of the blood glucose level (discussed below), and sends the reading to the control unit 20.

The communication unit 23 is connected, for example, to an external server device 30 (see FIG. 2), and sends and receives information about the patient stored in the server device 30 (such as the patient's medical history, medications being taken, risk level, age, diet, supplements, etc.). That is, on the basis of the information about the patient ID read by the barcode reader 22, for example, the communication unit 23 extracts the patient's medical history, medications being taken, risk level, age, diet, supplements, and other such information from the external server device 30 and sends this information to the control unit 20.

The battery 24 is, for example, a rechargeable battery, and is provided as the power supply of the biological information measurement device 10. The battery 24 drives the biological information measurement device 10 by supplying a substantially constant current to the control unit 20 and the like.

The memory unit 25 is a storage means for storing various kinds of data, and is connected to the control unit 20. The memory unit 25 stores, for example, blood glucose measurement results, patient information collated with the patient IDs read by the barcode reader 22, various messages used in processing for warning about blood glucose measurement (discussed below), and other such information.

With the biological information measurement device 10 in this embodiment, the server device that sends and receives data to and from the communication unit 23 stores various kinds of data, including information about the patient. When the patient ID read by the barcode reader 22 matches the patient data stored in advance in the memory unit 25, the control unit 20 controls the communication unit 23 so that information about the patient (such as the patient's medical history, medications being taken, risk level, age, diet, supplements, etc.) will be extracted from the server device 30 and received. The control unit 20 then determines the risk status of that patient by referring to the information about the patient received from the server device 30, and executes processing control to warn about blood glucose level measurement (discussed below).

### Effect of Dehydration on Blood Glucose Measurement

When the blood glucose level is measured in a patient showing symptoms of dehydration, error may skew the value higher. Therefore, when performing the measurement, it is preferable to give the measurer or the patient some kind of warning that the measured value is likely to include an error.

With the biological information measurement device 10 in this embodiment, the display of the display unit 14 is controlled so as to display, depending on the degree of dehydration, a message recommending that the patient consult with a doctor, a message recommending that the measurement be stopped, or a message recommending that the measurement be performed multiple times, or to display the measurement result as a reference value. These various messages are stored in the memory unit 25.

More specifically, the control unit 20 causes the display unit 14 to display, as a message to medical personnel (doctor or nurse) or the patient, a message notifying that there is a high probability that an error will occur in the blood glucose level measurement result.

In regard to dehydration, hyperglycemic hyperosmolar syndrome and diabetic ketoacidosis are known as symptoms related to diabetes, but in addition to these, the following are examples of medications and the like with which dehydration is apt to occur as various diseases, symptoms, and side effects.

### (a) Illnesses

diabetic ketoacidosis (DKA)
hyperosmolar hyperglycemia non-ketotic syndrome
hypotension
decompensated heart failure
peripheral arterial occlusive disease
diarrhea, vomiting (especially in young children)
fever (higher fever, more dehydrated)
cardiogenic: cardiopathy (shock (cardiogenic)/hypovolemic/anaphylactic:
   allergic reaction (anaphalactic)/septicemia: infection/toxic shock (septic)/neural: damage to the nervous system (neurogenic))
   (b) Symptoms (those that interfere with tissue perfusion)
tachycardia, tachysystole
confusion or diminished consciousness level
poor peripheral perfusion (cool, cyanosed extremities, poor capillary refill, poor peripheral pulses)
dysuria (poor urine output (<0.5 mL/kg/h))
metabolic acidosis
increased blood lactate concentration
   (c) Symptoms (those that interfere with tissue perfusion)
profuse sweating, moist skin
shallow breathing
pale, cool, clammy skin
chest pain
dizziness, light headedness or faintness
bluish lips or fingernails
anxiety
excessive sweating (vigorous exercise, hot weather)
increased urination (uncontrolled diabetes, diuretics, some BP medications)

### (d) Medications

vasodilators
beta blockers
ACE inhibitors
calcium channel blockers
Non-steroidal anti-inflammatory drugs (NSAIDs)
   (e) Other (age)
Infants and elderly people (more prone to dehydration)

### Flow of Processing to Warn About Blood Glucose Measurement

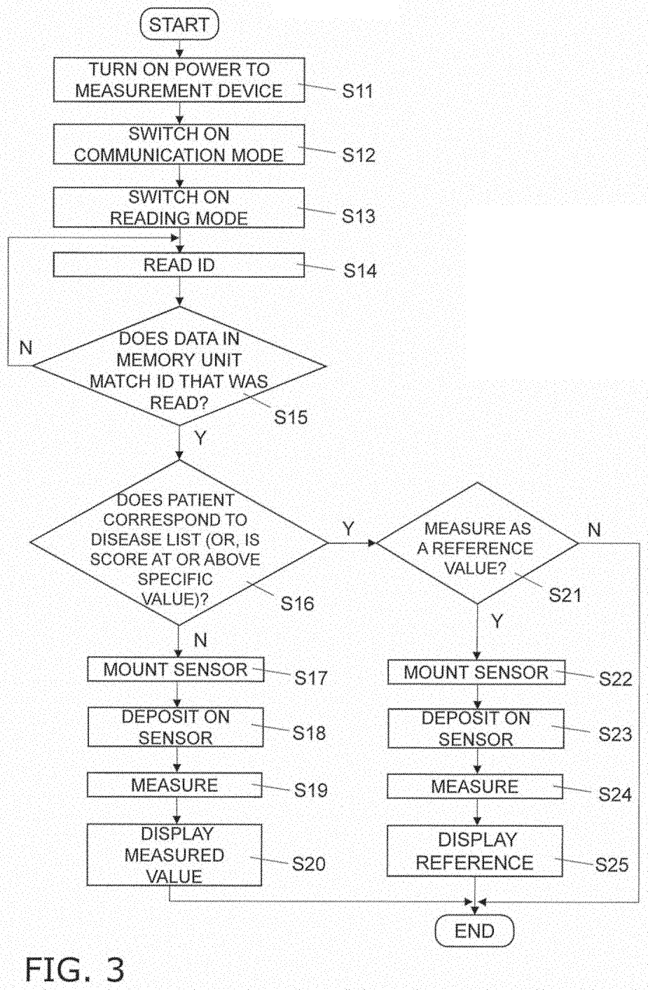

The biological information measurement device 10 in this embodiment performs processing to issue a warning about the measurement of the blood glucose level, on the basis of information about the patient, according to the flowchart shown in FIG. 3.

Specifically, in step S11, for example, the button 13 provided on the surface of the main body 11 of the biological information measurement device 10 is pressed and held to turn on the power.

Next, in step S12, the communication mode is switched on so that the communication unit 23 will be able to communicate with the external server device 30 and so forth.

Next, in step S13, the reading mode is switched on so that the barcode reader 22 will be able to read the barcode of the patient ID, etc.

Next, in step S14, the measurer (such as a nurse) reads the barcode by using the barcode reader 22 provided to the biological information measurement device 10. More specifically, the measurer uses the barcode reader 22 to acquire information about his or her own measurer ID, the patient ID, and the sensor lot number.

Here, the measurer ID is acquired by reading the barcode on the measurer's own name tag or the like with the barcode reader 22. The patient ID is acquired, for example, by reading the barcode on the surface of a band wrapped around the patient's wrist or the like with the barcode reader 22. The sensor lot number is acquired by reading the barcode on the outer peripheral surface of the container containing the blood glucose sensor inserted into the biological information measurement device 10 with the barcode reader 22.

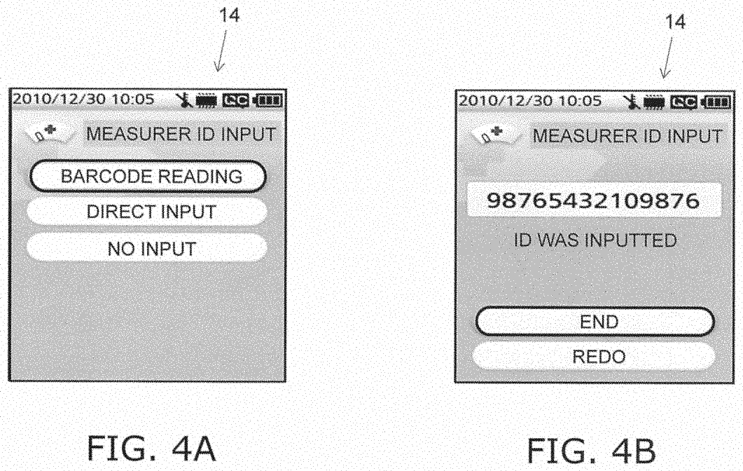

When the measurer ID is inputted, as shown on the display screen of the display unit 14 shown in FIG. 4A, one of three options is selected: read with the barcode reader 22, input directly, and do not input.

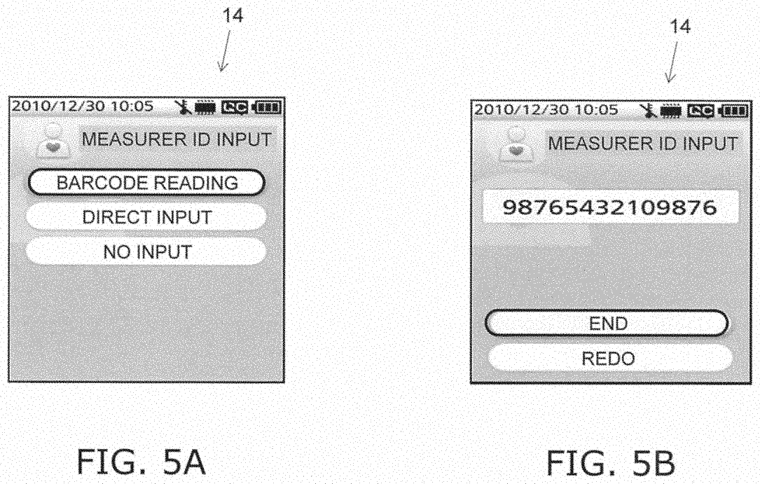

When “bar code reading” at the top is selected on the display screen shown in FIG. 4A and the measurer ID is read using the barcode reader 22, the measurer ID number that was read is displayed on the display screen of the display unit 14 as shown in FIG. 4B. Then, as shown in FIG. 4B, on the display screen of the display unit 14, the measurer selects whether to complete the work of inputting the measurer ID or to redo the input. If the measurer selects completion, the screen changes to the patient ID input screen shown in FIG. 5A.

When the patient ID is inputted, as shown on the display screen of the display unit 14 shown in FIG. 5A, one of three options is selected: read with the barcode reader 22, input directly, and do not input.

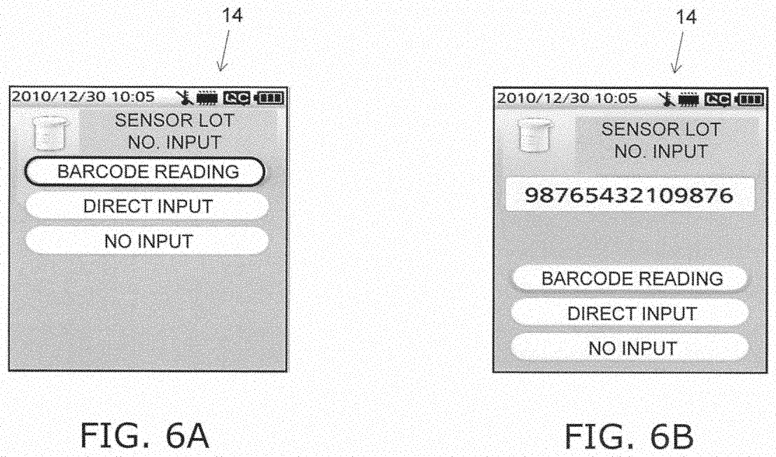

When “bar code reading” at the top is selected on the display screen shown in FIG. 5A and the patient ID is read using the barcode reader 22, the patient ID number that was read is displayed on the display screen of the display unit 14 as shown in FIG. 5B. Then, as shown in FIG. 5B, on the display screen of the display unit 14, the measurer selects whether to complete the work of inputting the patient ID or to redo the input. If the measurer selects completion, the screen changes to the sensor lot number input screen shown in FIG. 6A.

When the sensor lot number is inputted, as shown on the display screen of the display unit 14 shown in FIG. 6A, one of three options is selected: read with the barcode reader 22, input directly, and do not input.

When “bar code reading” at the top is selected on the display screen shown in FIG. 6A and the sensor lot number is read using the barcode reader 22, as shown in FIG. 6B, the sensor lot number that was read is displayed on the display screen of the display unit 14.

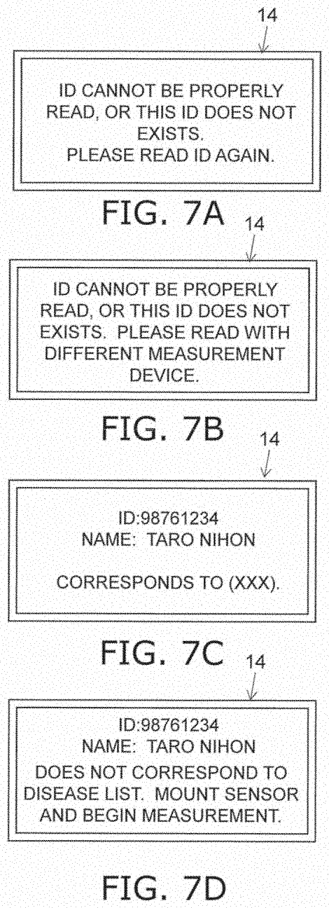

Here, if an ID cannot be read using the barcode reader 22, as shown in FIG. 7A, a message stating “The ID cannot be read properly or the corresponding ID does not exist. Read the ID again” is displayed on the display screen of the display unit 14.

Alternatively, as shown in FIG. 7B, a message stating “The ID cannot be read properly or the corresponding ID does not exist. Please try again with another measurement device” may be displayed on the display screen of the display unit 14.

Next, in step S15, it is determined whether or not the ID information acquired by the barcode reader 22 matches the information stored in the memory unit 25. That is, in step S15, the ID information for the patient whose blood glucose level is to be measured is compared to see if it matches the information of a hospitalized patient, for example.

Here, if the acquired ID information matches the information stored in the memory unit 25, the processing proceeds to step S16, and if there is no match, the processing returns to step S14.

The messages shown in FIGS. 7A and 7B may be displayed on the display screen of the display unit 14 as the result of the ID comparison in step S15.

Next, in step S16, using the information about the patient corresponding to the patient ID acquired in step S15, it is determined whether the patient corresponds to a certain disease list, or whether a score indicating the risk status of the patient is at or above a certain value.

That is, in step S16, the information about the patient corresponding to the patient ID acquired in step S14 is received from the external server device 30 via the communication unit 23, and is compared with a disease list or the like.

The information about the patient obtained through the communication unit 23 includes, for example, one or more of the following: the patient's medical history, medications being taken, current symptoms, risk level, age, diet, and supplements.

If the patient does not correspond to the disease list or the like, the processing proceeds to step S17, and if the patient does correspond, the processing proceeds to step S21.

If the patient ID reading is normal in step S14, and the patient corresponds to a certain disease list or the like in step S16, then the display screen of the display unit 14 displays information such as the patient ID number, the patient name, the corresponding medical history and so forth, as shown in FIG. 7C.

On the other hand, if the patient does not correspond in step S16, as shown in FIG. 7D, then that patient does not correspond to the patient ID number, the patient name, and the disease list, so the display screen of the display unit 14 displays a message to the effect that normal measurement can be performed. This message indicating that normal measurement is possible shown in FIG. 7D may be omitted.

Next, in step S17, the sensor 1 is mounted in the sensor insertion port 12 of the biological information measurement device 10 in order to carry out normal measurement.

Next, in step S18, the blood B that comes out after a puncture is made in the patient's finger F1 using a puncture device (not shown) is deposited into the deposition port 1a of the sensor 1 inserted into the sensor insertion port 12 of the biological information measurement device 10.

Next, in step S19, if the patient's blood B has been normally deposited in step S18, then the blood glucose level is automatically measured by the measurement unit 21 of the biological information measurement device 10.

Next, in step S20, the measured blood glucose level, which is the result of the measurement in step S19, is displayed on the display screen of the display unit 14. This concludes normal blood glucose measurement.

Next, in step S21, since it was determined in step S16 that the patient corresponded to a certain disease list, or that the score was at or above a certain value, it is assumed that the measurement result will include a large error if the blood glucose level is measured. Consequently, in step S21, the measurer confirms whether or not to measure the blood glucose level as a reference value.

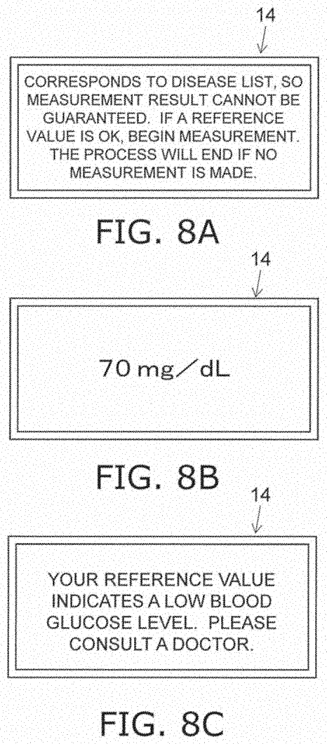

More specifically, as shown in FIG. 8A, the display screen of the display unit 14 displays a message that “This patient corresponds to disease list, so the measurement result cannot be guaranteed. Go ahead and measure, but only as a reference value. If no measurement is performed, this process will be terminated.”

Here, if the user wishes to carry out measurement as a reference value, the processing proceeds to step S22. On the other hand, if the user does not wish to carry out measurement as a reference value, the processing is terminated without going any further.

Next, in step S22, the sensor 1 is mounted in the sensor insertion port 12 of the biological information measurement device 10 in order to measure the blood glucose level as a reference value.

Next, in step S23, the blood B that has come out of the puncture wound on the patient's finger F1 made with the puncture device (not shown) is deposited in the deposition port 1a of the sensor 1 inserted into insertion port 12 of the biological information measurement device 10.

Next, in step S24, if the patient's blood B has been properly deposited in step S23, the blood glucose level is automatically measured by the measurement unit 21 of the biological information measurement device 10.

Next, in step S25, the blood glucose level measured in step S24 is displayed as a reference value on the display screen of the display unit 14. This concludes the measurement of blood glucose level as a reference value.

At this point, the display screen of the display unit 14 displays the numerical value of the measurement result shown in FIG. 8B (here, 70 mg/dL), as well as a message recommending that the user consult with a doctor, stating “Your reference value is at a hypoglycemic level. Please consult your doctor,” as shown in FIG. 8C, according to the measurement result.

As described above, the biological information measurement device 10 in this embodiment determines whether to carry out normal measurement or to carry out measurement as a reference value, on the basis of the information about the patient acquired from the external server device 30 via the communication unit 23, and the display screen of the display unit 14 displays a message indicating whether or not to carry out measurement as a reference value, etc.

More specifically, in this embodiment, on the basis of information that could affect the result of measuring blood glucose level, such as the patient's medical history, medications being taken, current symptoms, risk level, age, diet, supplements, etc., a warning is issued about the measurement of blood glucose level, such as whether or not to carry out the measurement as a reference value.

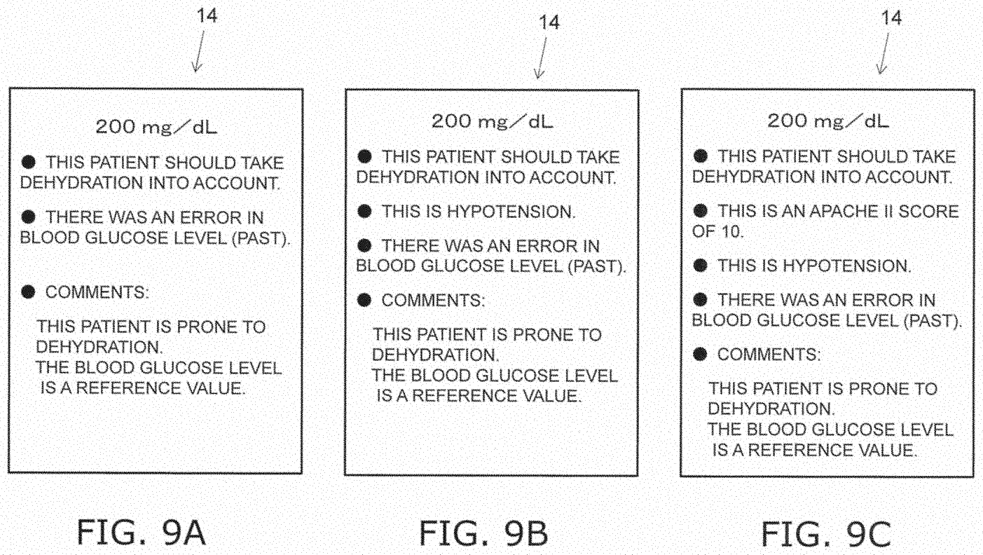

Consequently, as shown in FIG. 9A, depending on the information about the patient, the display screen of the display unit 14 displays the measurement result of 200 mg/dL and messages that “This patient should take dehydration into account” and “There was an error in blood glucose level (past),” along with the comment that “This patient is prone to dehydration. The blood glucose level is a reference value,” thereby alerting the measurer to take caution.

Alternatively, as shown in FIG. 9B, the display screen of the display unit 14 displays the measurement result of 200 mg/dL and the messages that “This patient should take dehydration into account,” “This is hypotension,” and “There was an error in blood glucose level (past),” along with the comment that “This patient is prone to dehydration. The blood glucose level is a reference value,” thereby alerting the measurer to take caution.

Furthermore, as shown in FIG. 9C, the display screen of the display unit 14 displays the measurement result of 200 mg/dL and the messages that “This patient should take dehydration into account,” “This is an APACHE II score of 10,” “This is hypotension,” and “There was an error in blood glucose level (past),” along with the comment that “This patient is prone to dehydration. The blood glucose level is a reference value,” thereby alerting the measurer to take caution.

### Embodiment 2

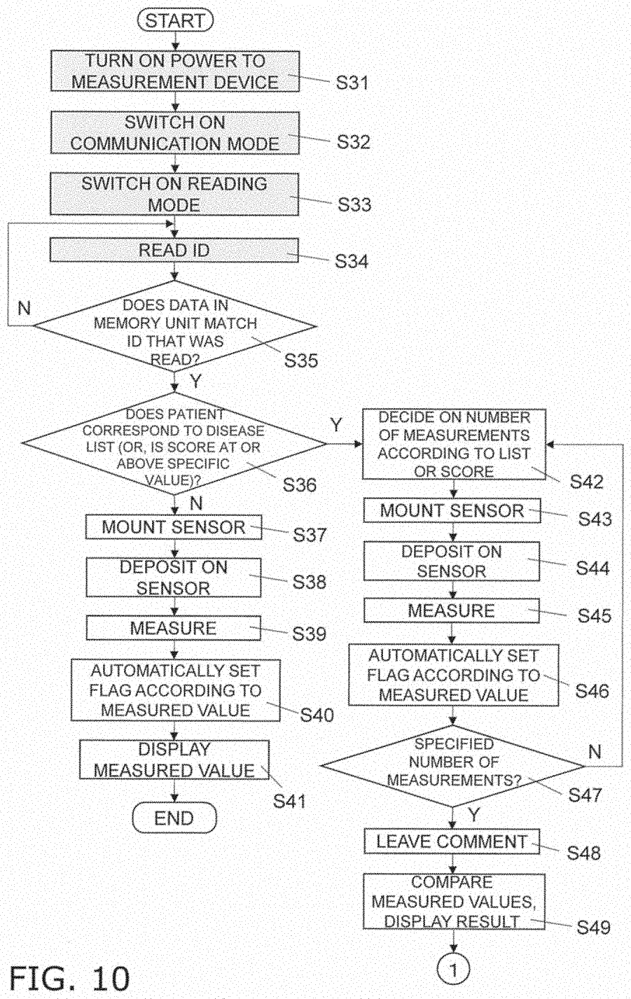

The flow of the process for warning about measurement of blood glucose level in the biological information measurement device 10 according to another embodiment of the present invention will be described below with reference to FIGS. 10 to 12B.

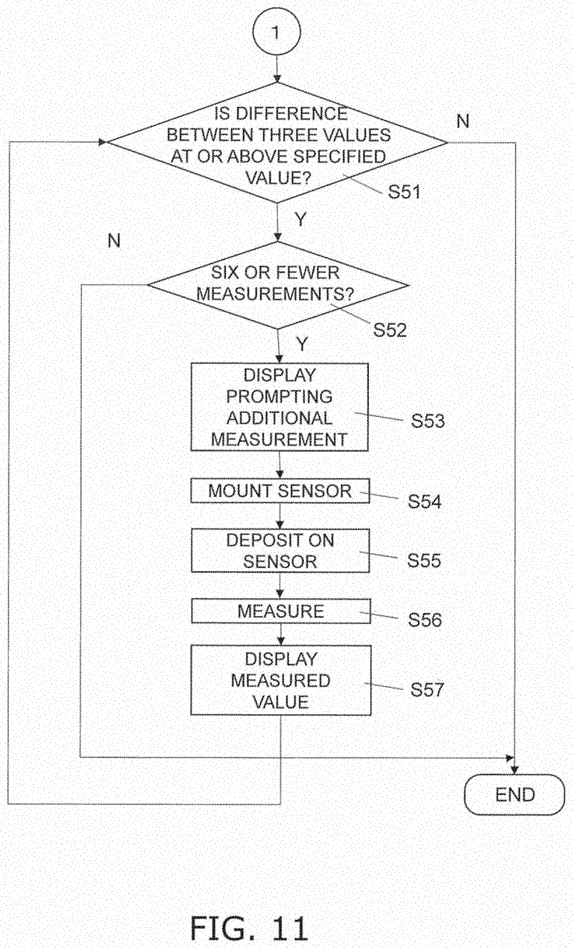

That is, the biological information measurement device 10 in this embodiment has the same configuration as the biological information measurement device 10 in the first embodiment, and performs processing for warning about measurement of blood glucose level according to the flowcharts shown in FIGS. 10 and 11.

That is, as shown in FIG. 10, in step S31, for example, the button 13 provided on the surface of the main body 11 of the biological information measurement device 10 is pressed and held to turn on the power.

Next, in step S32, the communication mode is switched on so that the communication unit 23 will be able to communicate with the external server device 30 and the like.

Next, in step S33, the reading mode is switched on so that the barcode reader 22 will be able to read the barcode of the patient ID, etc.

Next, in step S34, the measurer (such as a nurse) reads the barcode by using the barcode reader 22 provided to the biological information measurement device 10. More specifically, the measurer uses the barcode reader 22 to acquire information about his or her own measurer ID, the patient ID, and the sensor lot number.

Since the display example on the display screen of the display unit 14 during ID reading is the same as that in the first embodiment given above, it will not be described again here.

Next, in step S35, it is determined whether or not the ID information acquired by the barcode reader 22 matches the information stored in the memory unit 25. That is, in step S35, a comparison is performed to see whether or not the ID information of the patient whose blood glucose level is to be measured matches the information of a hospitalized patient, for example.

Here, if the acquired ID information matches the information stored in the memory unit 25, the processing proceeds to step S36, and if there is no match, the processing returns to step S34.

Next, in step S36, information about the patient corresponding to the patient ID acquired in step S35 is used to determine whether this patient corresponds to a certain disease list, or a score indicating the risk status of the patient is at or above a specific value.

That is, in step S36, the information about the patient corresponding to the patient ID acquired in step S34 is received from the external server device 30 via the communication unit 23, and compared with a certain disease list or the like.

The information about the patient obtained through the communication unit 23 includes, for example, one or more of the following: the patient's medical history, medications being taken, current symptoms, risk level, age, diet, and supplements.

Here, if the patient does not correspond to a disease list or the like, the processing proceeds to step S37, but if there is any match, the processing proceeds to step S42.

Next, in step S37, the sensor 1 is mounted in the sensor insertion port 12 of the biological information measurement device 10 in order to carry out normal measurement.

Next, in step S38, the blood B that has been brought out of a puncture wound on the patient's finger F1 using a puncture device (not shown) is deposited in the deposition port 1a of the sensor 1 inserted into the sensor insertion port 12 of the biological information measurement device 10.

Next, in step S39, if the patient's blood B is properly deposited in step S38, the blood glucose level is measured automatically by the measurement unit 21 of the biological information measurement device 10.

Next, in step S40, if the measurement result is higher or lower than a specific value, for example, a flag corresponding to the measured value is automatically set so that a subsequent search for that measurement result will be possible.

Next, in step S41, the blood glucose level measured in step S39 is displayed on the display screen of the display unit 14. This concludes the normal blood glucose measurement.

Next, in step S42, since it was determined in step S36 that the patient corresponded to a certain disease list, or that the score was at or above a certain value, it is assumed that the measurement result will include a large error even if the blood glucose level is measured. Consequently, in step S42, in order to average measurement results in which error is likely to occur, the number of measurements is decided toward increasing the number of measurements according to the risk status of the patient.

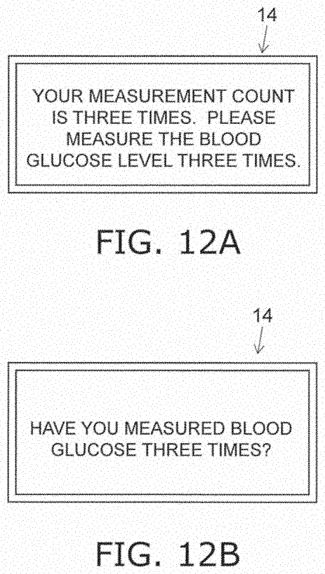

More specifically, as shown in FIG. 12A, a message stating “Your measurement count is three times. Please measure the blood glucose level three times” is displayed on the display screen of the display unit 14.

Next, in step S43, the sensor 1 is mounted in the sensor insertion port 12 of the biological information measurement device 10 in order to measure the blood glucose level.

Next, in step S44, the blood B that has been brought out of a puncture wound in the patient's finger F1 using a puncture device (not shown) is deposited into the deposition port 1a of the sensor 1 inserted into the sensor insertion port 12 of the biological information measurement device 10.

Next, in step S45, if the patient's blood B is properly deposited in step S44, the blood glucose level is automatically measured by the measurement unit 21 of the biological information measurement device 10.

Next, in step S46, just as in step S40, if the measurement result is at or above a certain value, for example, a flag corresponding to the measured value is automatically set so that a subsequent search for that measurement result will be possible.

Next, in step S47, it is determined whether or not the specified number of measurements has been reached.

At this point, as shown in FIG. 12B, a message reading “Has the blood glucose level been measured three times?” is displayed on the display screen of the display unit 14.

In addition, the display screen of the display unit 14 may display messages indicating which measurement this is, namely, “First measurement,” “Second measurement,” and “Third measurement.”

Here, if the specified number of times (such as three times) has been reached, the processing proceeds to step S48, and otherwise the processing returns to step S42.

Next, in step S48, since measurement the specified number of times has been completed, a comment corresponding to the measurement result is added.

Next, in step S49, the measurement results in step S45 are compared, and the measurement results of three times are displayed on the display screen of the display unit 14. This concludes the three blood glucose measurements.

Furthermore, as shown in FIG. 11, in step S51, it is determined whether or not the difference between the three measurement results (three values) is at or above a certain value. That is, the degree of variance in the three measured values is checked.

Here, if the difference at or over a specific value, that is, if there is a large amount of variance in the three values, the processing proceeds to step S52, but if the difference is not at or above a certain value, that is, if there is little variance in the three values, the results of the three values are taken to be the final measured values, and the processing is ended.

Next, in step S52, it is determined whether or not the number of measurements is within the maximum number of times (six times in this embodiment). Here, if the maximum number of times (six times) has been reached, the three values having a large variance are taken as the final measured values, and the processing is ended. On the other hand, if the maximum number of times (six times) has not been reached, the processing proceeds to step S53.

Next, in step S53, a message recommending additional measurement is displayed on the display screen of the display unit 14.

Next, in step S54, the sensor 1 is mounted in the sensor insertion port 12 of the biological information measurement device 10 in order to measure the blood glucose level for the additional measurement.

Next, in step S55, the blood B that has been brought out of a puncture wound in the patient's finger F1 using a puncture device (not shown) is deposited into the deposition port 1a of the sensor 1 inserted in the sensor insertion port 12 of the biological information measurement device 10.

Next, in step S56, if the patient's blood B is normally deposited in step S55, the blood glucose level is automatically measured by the measurement unit 21 of the biological information measurement device 10.

Next, in step S57, the measurement result for the additional measurement is displayed on the display screen of the display unit 14.

After this, the processing again returns to step S51, and the processing of steps S51 to S57 is repeated until the variance in the three values is within a specified value, or until the maximum number of times is reached in step S52.

The number of measurements set in step S51 is not limited to three, and should be set as follows, according to information about the patient (the patient's medical history, current symptoms, medications being taken, the patient's risk level, age, diet, supplements, etc.).

### (i) Medical History

One measurement: none
Two measurements: diarrhea, general heart disease (cardiogenic), acute allergic reaction (anaphalactic), general nerve disease (neurogenic), hypothermia, metabolic acidosis, increased blood lactate concentration
Three or more measurements: general hypovolemic disease (hypovolemic), sepsis (septic)

### (ii) Symptoms, Age, etc.

One measurement: none
Two measurements: infants and the elderly, high fever, hyperhidrosis (all of these are inpatients who are prone to dehydration), anxiety, bluish lips and nails, chest pain, dizziness, light-headedness, weakness, pale face, cold sweats, tachycardia, urine output reduced to <0.5 ml/kg/h, weakened pulse
Three or more measurements: infants with vomiting and diabetic patients whose blood glucose level cannot be controlled (both of these are inpatients who are dehydrated)
   (iii) Medications
One measurement: none
Two measurements: acetaminophen, ascorbic acid (vitamin C), infusions containing sugars such as maltose, xylose, and galactose, uric acid, bilirubin, and the like, which are recognized as existing interfering substances
Three or more measurements: vasodilators, beta blockers, ACE inhibitors, calcium channel blockers, NSAIDs (all of these are medications that reduce blood flow), and diuretics (medications that cause dehydration)
   (iv) Risk level (APACHE II: predicted mortality rate of 50% at a score of 25, and 90% at 36 or higher)
One measurement: score of at least 0 but less than 25
Two measurements; score of at least 25 but less than 36
Three or more measurements: score of at least 36, maximum score of 71
   (v) Risk level (MODS: predicted in-patient mortality rate of 50% at a score of 12, 82% at 20 or more, and 100% at 21 or more)
One measurement: score of at least 0 but less than 12
Two measurements: score of at least 12 but less than 20
Three or more measurements: score of at least 20, maximum score of 24

Also, in the processing in steps S51 to S57, if there is a great deal of variance in the three values, such as when two of the three measurements are about 150 mg/dL and one is 250 mg/dL, one additional measurement is performed.

Then, if the fourth measurement result of the additional measurement is 250 mg/dL, one more additional measurement is performed, and if the fifth measurement result is 250 mg/dL, the average measurement value should be 250 mg/dL.

### OTHER EMBODIMENTS

An embodiment of the present invention was described above, but the present invention is not limited to or by the above embodiment, and various modifications are possible without departing from the gist of the invention.

### (A)

In the above embodiment, an example was given in which the communication unit 23 that communicated with the server device 30 in which the information about the patient was stored was used as the information acquisition unit for acquiring information about the patient, but the present invention is not limited to this.

For instance, instead of a communication unit, the information acquisition unit for acquiring information about the patient can also be an input reception unit that acquires information about the patient that has been manually inputted by the measurer or the patient himself

Furthermore, if the information about the patient is stored in the memory unit 25 in the biological information measurement device 10, the control unit 20 may function as an information acquisition unit.

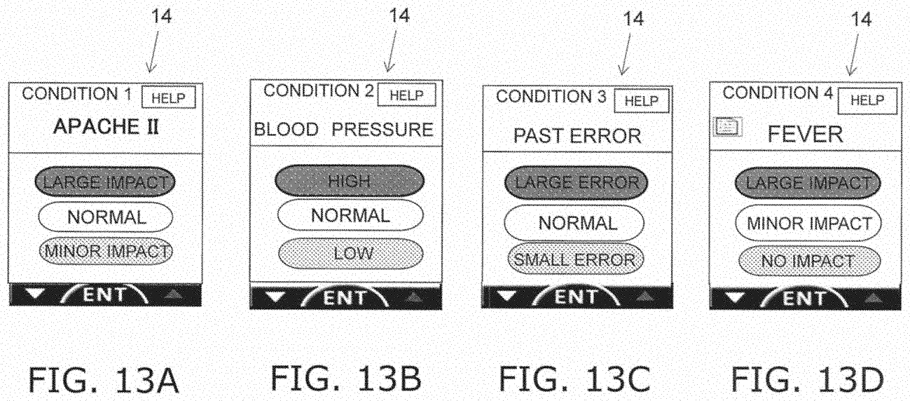

Here, when the measurer or the patient directly inputs information about the patient (medical history, symptoms, medications, etc.), the required information should be inputted on the display screen of the display unit 14 shown in FIGS. 13A to 13D.

For example, as shown in FIG. 13A, when an APACHE II score is inputted as information about a patient, the information is inputted by selecting one of three evaluation levels (large impact, normal, minor impact) set by a doctor according to the patient.

The three-level evaluation of APACHE II displayed here should be, for example, within the range of the average APACHE II values for the last two or three days.

As shown in FIG. 13B, when blood pressure is inputted as information about the patient, the information is inputted by selecting one of three evaluation levels (high, normal, low) to which the numerical value of the measurement result set in advance by a doctor belongs.

Also, as shown in FIG. 13C, when an error in a past measurement value is inputted as information about the patient, the information is inputted by selecting one of three evaluation levels (large error, normal, small error) to which the numerical value of the error range set in advance by a doctor belongs.

For example, large error is usually selected if the past error is ±50 mg/dL or more, normal error if it is ±20 mg/dL or more, and small error if it is less than ±10 mg/dL.

Furthermore, as shown in FIG. 13D, when the current symptoms (fever) are inputted as information about the patient, the information is inputted by selecting one of three evaluation levels (large impact, minor impact, no impact) to which the body temperature set in advance by a doctor belongs.

### (B)

In the above embodiment, an example was given in which information about the patient (the patient's medical history, medications being taken, current symptoms, risk level, and age) was acquired a server device 30 provided outside the biological information measurement device 10, via the communication unit 23. However, the present invention is not limited to this.

For example, control may be performed so that information about the patient is stored in a memory unit provided inside the biological information measurement device, and is extracted when measurement is performed.

### (C)

In this embodiment, an example was given in which warning processing was performed by taking into account the degree of dehydration as a risk factor that may include an error in the blood glucose level measurement result, but the present invention is not limited to this.

For instance, warning processing may be performed by taking into account other factors that affect the measurement of blood glucose level as risks.

Also, processing to warn about the measurement of biological information other than blood glucose level may be performed by taking into account various factors that affect the measurement as risks.

### (D)

In the second embodiment, an example was given in which, when the risk level of the patient was determined to be high on the basis of information about the patient, the patient was encouraged to perform three measurements, and to repeat measurements, up to a maximum of six times, until the maximum variance in the measured values is within the specified range. However, the present invention is not limited to this.

For example, the plurality of measurements may be two times, or four or more times, depending on the risk level of the patient and the measurement of various biological information. Also, as to the maximum number of times, the maximum number of times need not be set, or may be set to five or fewer times, or to seven or more times.

Also, when increasing the number of measurements, rather than squeezing out the blood from the initial puncture site (such as a fingertip), it is preferable to change to a new puncture site and squeeze out the blood from there.

### (E)

In the above embodiment, an example was given in which the server device 30 was used as an external storage means for sending and receiving various kinds of data, including information about the patient, to and from the communication unit 23, but the present invention is not limited to this.

For instance, instead of a server device, a cloud space may be used as an external storage means.

### (F)

In the above embodiment, an example was given in which the patient's medical history, medications being taken, current symptoms, risk level, and age were used as information about the patient, but the present invention is not limited to this.

For example, one or more of the following types of information may be used as information about the patient, the patient's medical history, medications being taken, current symptoms, risk level, age, diet, supplements, etc., or some other information besides these may be used as information about the patient.

### (G)

In the above embodiment, an example was given in which text information (message) was displayed on the display unit 14 as a warning about the measurement of biological information based on the information about the patient, but the present invention is not limited to this.

For instance, instead of text information, a voice message, a warning sound, or the like may be outputted from an output unit (speaker) that outputs audio information.

Also, it is possible, for example, to issue a warning consisting of optical information in various colors, such as red, green, or blue, with an LED (light emitting diode) or the like.

Alternatively, text information, audio information, and optical information may be used in combination.

### (H)

In the above embodiment, an example was given in which a blood glucose meter for measuring blood glucose level was used as the biological information measurement device according to the present invention, but the present invention is not limited to this.

For instance, the present invention may be applied to a biological information measurement device that measures biological information other than blood glucose level (ketone level, total cholesterol level, HDL cholesterol level, LDL cholesterol level, triglyceride level, uric acid level, albumin level, AST (GOT) value, ALT (GPT) value, ALP value, LDH value, LAP value, CHE value, HBs value, HCV value, amylase value, HbA1c value, urinary glucose level, urea nitrogen level, creatinine value, eGFR value, urinary protein value, urinary occult blood level, urinary pH value, sodium (Na) value, potassium (K) value, chlorine (Cl) value, RF value, lipoprotein value, triglyceride value, etc.).

An example of another biological information measurement device is a biosensor that makes use of an antigen-antibody reaction, such as a gold colloidal immuno-chromatographic measurement system for measuring CRP.

This biosensor comprises an addition portion for adding the solution to be tested, and is made up of a plurality of development layers through which the solution to be tested can permeate, wherein an anti-CRP antibody is immobilized in a dry state ahead of time on some of the development layers. Furthermore, the configuration is such that an anti-CRP antibody that is labeled with colloidal gold (this does not apply to CRP, which is a pentamer) and with which it is possible to bind to CRP with an epitope different from that of the immobilized antibody that is further on the upstream side of the permeation of the solution to be tested during measurement than the antibody-immobilized portion, is held in advance in a dry state that allows for elution by the solution to be tested. During measurement, if the required amount of the solution to be tested is added to the added portion, the solution to be tested permeates the development layers and the measurement is commenced. If the solution to be tested is whole blood, the blood cell component is filtered or hemolyzed, or the solution permeates the development layers downstream through capillary action while simultaneously developing with a liquid component such as plasma, reaching the labeled antibody portion, the labeled antibody is eluted, and the CRP in the test solution and the labeled anti-CRP antibody react with each other and permeate downstream. After this, the solution reaches the immobilized anti-CRP antibody portion, and an antigen-antibody reaction with the immobilized antibody further occurs to form a triple complex of immobilized antibody-CRP-labeled antibody according to the CRP concentration. The labeled antibody that did not form a complex permeates further downstream. Consequently, only the labeled antibody that has formed a triple complex is captured in the immobilized antibody portion. The antibody-immobilized portion is irradiated with light having a wavelength corresponding to the labeled gold colloidal particle shape and the reflected absorbance thereof is sensed, which allows the bound labeled antibody to be detected and CRP to be measured.

Here, an example was described in which the measurement principle was based on a sandwich reaction in an antigen-antibody reaction, but various other antigen-antibody reactions can be conducted on a biosensor, such as using an antigen or the like in the immobilized region, or using a measurement principle based on a competitive reaction in which a labeled antigen is labeled rather than an antibody. In addition to colloidal gold, the labeled substance can also be latex particles, a fluorescent dye, or an enzyme, which allow optical detection of an enzyme reaction at the immobilized portion.

An optical reading device can similarly measure biological information by reading reflected light, transmitted light, fluorescent light, etc., or by using a camera to capture data as an image, and subjecting the image to computation processing to detect the labeled substance of the immobilized portion. Furthermore, although CRP measurement was used as an example here, by changing the antibody design from a property whose basic principle is an antigen-antibody reaction, other measurement categories can be handled with the same configuration, such as troponin I, troponin T. CK-MB, myoglobin, heart-derived fatty acid binding protein (H-FABP), D-dimer, BNP, and NP-proBNP, which are useful markers for early diagnosis of acute myocardial infarction, exclusion tests for pulmonary thromboembolism, and the monitoring of patients with heart failure, the measurement of procalcitonin (PCT). HBs antigen, HBs antibody, HCV antibody, HIV antibody, HIV antigen, HIV antibody, syphilis TP antibody, candida antigen, etc., which are markers for infectious diseases, septicemia, and inflammation tests, the measurement of HbA1c and glycoalbumin, which are markers in testing for diabetes, and the measurement of specific IgE, etc., which are used in allergy tests.

Yet another example of a biological information measurement device is a biosensor that does not measure a specific biomarker, but makes use of a tissue factor in the reagent layer on a sensor, an example of which is an electrochemical prothrombin time measurement system that measures prothrombin time.

This biosensor comprises an addition portion for adding a test solution, and is made up of electrodes and a reagent layer on which the test solution reacts. The reagent layer contains a tissue factor and a substrate electrozyme TH in a dry state in advance.

During measurement, when the required amount of the test solution is added to the addition portion, the test solution dissolves the reagent layer and the test is commenced. The tissue factor contained in the reagent layer activates an exogenous coagulation mechanism to produce thrombin from the prothrombin in the sample, and the thrombin thus generated decomposes the electrozyme TH contained in the reagent layer to produce phenylenediamine.

When voltage is applied to two or more pairs of electrodes near the reagent layer, phenylenediamine is oxidized to generate an electric current, how long it takes for the electric current to reach a threshold value is measured, and the prothrombin time in the sample can be measured.

Here, an example was given in which the measurement principle was based on an electrochemical prothrombin time measurement system, but it is also possible, for example, to optically detect, either directly or indirectly, the change in viscosity attributable to an extrinsic coagulation activation mechanism.

An optical reading device can similarly measure biological information by reading reflected light, transmitted light, fluorescent light, etc., or by using a camera to capture data as an image and subjecting the image to computation processing to sense the optical change in the reagent layer. Furthermore, although the prothrombin time was used as an example here, it is also possible to measure the prothrombin time with the same reagent layer configuration by changing the detection system from a property in which the basic principle is the measurement of the change in viscosity to the electrochemical measurement of electrical resistance, measurement of magnetic substance mobility, etc.

Conversely, the intrinsic coagulation activation mechanism can be measured with various kinds of detection system by changing the reagent layer to an activated substance such as eladic acid.

That said, the prothrombin time affects the condition and disease of the liver, which makes coagulation factors, vitamin K-containing products and supplements that affect coagulation factors, and cytochrome P450 drug-metabolizing enzyme systems, and measured values may also be affected by various factors such as drugs that interact with warfarin.

Patients who need to measure prothrombin time are mainly those taking warfarin, but drugs that interact with warfarin include acarbose, acetaminophen, allopurinol, amiodarone, amprenavir, acetylsalicylic acid (ASA), atazanavir, azathioprine and mercaptopurine, azithromycin, bismuth subsalicylate, bosentan, carbamazepine (CBZ), celecoxib, cholestyramine, cimetidine, ciprofloxacin, clarithromycin, clopidogrel, cloxacillin, colchicine, danazol, darunavir, delavirdine, diclofenac, disopyramide, doxycycline, dronedarone, efavirenz, erythromycin, ethinyl estradiol, etravirine, fenofibrate, fluconazole, fluvastatin, fosamprenavir, gemfibrozil, glyburide, ibuprofen, indinavir, indomethacin, isoniazid, isotretinoin, itraconazole and ketoconazole, lactulose, lansoprazole, leflunomide, levofloxacin, levothyroxine, Kaletra (lopinavir/ritonavir), mesalamine, methimazole, topical methyl salicylate, metronidazole, miconazole (oral, topical, or vaginal formulation), moxifloxacin, naproxen, nelfinavir, nevirapine, omeprazole, orlistat, phenobarbital, phenytoin, predonisone, propafenone, propoxyphene, propylthiouracil, quetiapine, raloxifene, ranitidine, ribavirin, rifampin, ritonavir, ropinirole, rosuvastatin, saquinavir, simvastatin, sulfamethoxazole (with or without trimethoprim), sulfamethoxazole (with or without trimethoprim), sulfasalazine, sulfinpyrazone, terbinafine, tetracycline, ticlopidine, tipranavir, tramadol, voriconazole, etc.

Macrolide antibiotics, cimetidine, and flucanzole are known medications that affect the cytochrome P450 drug-metabolizing enzyme system.

Vegetables containing vitamin K, which affects coagulation factors, include asparagus, beans, beets, broccoli, Brussels sprouts, cabbage, collards, cucumbers, dandelions, endive, kale, lettuce, mustard, okra, onion, parsley, peas, rhubarb, soybeans, spinach, turnip greens, peanuts, corn, safflower, walnuts, sesame, olives, canola oil, etc. These are found in fast foods and processed foods, such as cheeseburgers, hamburgers with sauce, chicken sandwiches, fish sandwiches, French fries, beef tacos, Cheeto-type chips, potato chips, olestra potato chips, tortilla chips, olestra tortilla chips, etc. These are also found in traditional Japanese foods, such as algae-purple laver, algae-konbu, algae-hijiki, asatsuki leaf, ashitaba leaf, bok-choy, komatsuna, pistachio, etc.

Nutritional supplements containing coumarin derivatives include alfalfa, angelica root, aniseed, amica, artemesia, Asa foetica (asafoetida), bishop's weed, bogbean, buchu, capsicum, cassia, celery seed, chamomile, danshen (Salvia miltiorrhiza), dandelion, dong quai (danggui, Angelica sinensis), fenugreek, horse chestnut, horseradish, licorice root, lovage root, meadow sweet, melilot, nettle, parsley, passion flower, prickly ash, quassia, red clover, rue, sweet clover, sweet woodruff, tonka beans, wild carrot, wild lettuce, etc.

Dietary supplements that may reduce the absorption of warfarin include agar, algin, aloe, barley, blond psyllium, butternut, carrageenan, cascara, castor, coffee charcoal, European buckthorn, Iceland moss, glucomannan, jalap, karaya gum, larch arabinogalactan, marshmallow, Mexican scammony root, quince, rhubarb, rice bran, slippery elm, tragacanth, etc.

Dietary supplements that affect the chitochrome P450 drug-metabolizing enzyme system include bergamotin (a component of grapefruit juice), bishop's weed (bergapten), bitter orange, cat's claw, chrysin, cranberry, devil's claw, dehydroepiandrosterone, diindolymethane, echinacea, eucalyptus, feverfew, fo-ti, garlic, ginseng, goldenseal, guggul, grape, grapefruit juice, indole-3-carbinol, ipriflavone, kava, licorice, lime, limonene, lycium (Chinese wolfberry), milk thistle, peppermint, red clover, resveratrol, St. John's wort, sulforaphane, valerian, wild cherry, etc.

Other conditions that affect a patient's metabolism include dehydration, chronic inflammatory disease, thyroid disease, fever, pregnancy, chronic fibrinogen elevation, late-stage cancer, and dialysis treatment for end-stage renal disease. Drugs that can have an effect include almost all antibiotics, acetaminophen, aspirin, and non-steroidal anti-inflammatory drugs, and it is clinically very useful to utilize information about a patient when measuring various samples.

### INDUSTRIAL APPLICABILITY

The biological information measurement device of the present invention has the effect of allowing a warning about the measurement of biological information to be issued on the basis of information such as the medical history and symptoms of the patient and any medications being taken, and therefore can be widely applied to various devices that measure biological information.

### REFERENCE SIGNS LIST

1 sensor
1a deposition port
1b electrode portion
10 biological information measurement device
11 main body
12 sensor insertion portion (sensor mounting portion)
13 button
14 display unit
20 control unit
21 measurement unit
22 barcode reader (reading unit)
23 communication unit (information acquisition unit)
24 battery
25 memory unit
30 server device
B blood
F1 finger

1. A biological information measurement device, comprising:
a sensor mounting unit to which is mounted a sensor for measuring biological information about a patient;
a measurement unit that is connected to the sensor mounted to the sensor mounting unit, and configured to measure the biological information;
an information acquisition unit configured to acquire information about the patient for whom the biological information is measured; and
a control unit configured to determine a risk status of the patient on the basis of the information about the patient acquired by the information acquisition unit, and issue a warning about a measurement of biological information about the patient with the measurement unit on the basis of a determination result.

2. The biological information measurement device according to claim 1,
wherein the information about the patient includes one or more of the following: a medical history of the patient, medications being taken, current symptoms, risk level, age, diet, and supplements.

3. The biological information measurement device according to claim 1,
further comprising a memory unit configured to store information about the patient.

4. The biological information measurement device according to claim 1,
further comprising a communication unit configured to transmit and receive information about the patient to and from an external device.

5. The biological information measurement device according to claim 4,
wherein the communication unit transmits and receives information about the patient to and from a server device provided externally.

6. The biological information measurement device according to claim 1,
further comprising a display unit configured to display a measurement result from the measurement unit.

7. The biological information measurement device according to claim 6,
wherein the control unit controls the display unit so as to display to a measurer a message asking whether or not to measure the biological information of the patient, on the basis of the determination result.

8. The biological information measurement device according to claim 6,
wherein the control unit controls the display unit so as to display to a measurer a message telling to measure the biological information of the patient as a reference value, on the basis of the determination result.

9. The biological information measurement device according to claim 6,
wherein the control unit controls the display unit so as to display to a measurer the number of measurements when measuring the biological information about the patient, on the basis of the determination result.

10. The biological information measurement device according to claim 1,
further comprising a reading unit configured to read information about the patient, a measurer, and the sensor.

11. A biological information measurement method, comprising:
an information acquisition step of acquiring information about a patient whose biological information is to be measured;
a determination step of determining a risk status of the patient on the basis of the information about the patient acquired in the information acquisition step; and
a warning step of issuing a warning about a measurement of biological information about the patient, on the basis of a determination result in the determination step.

12. The biological information measuring method according to claim 11,
wherein, in the warning step, a message asking whether or not to measure biological information about the patient is displayed on the display unit to a measurer on the basis of the determination result in the determination step.

13. The biological information measuring method according to claim 11,
wherein, in the warning step, a message telling to measure the biological information about the patient as a reference value is displayed on the display unit to a measurer on the basis of the determination result in the determination step.

14. The biological information measuring method according to claim 11,
wherein, in the warning step, the display unit displays the number of measurements when measuring the biological information about the patient to a measurer, on the basis of the determination result in the determination step.
